(19) United States
(12) Patent Application Publication
(10) Pub. No.: US 2017/0027608 A1
(43) Pub. Date: Feb. 2, 2017

(54) AUTOMATED INSERTION AND EXTRACTION OF AN IMPLANTED BIOSENSOR

(71) Applicant: Optoelectronics Systems Consulting, Inc., Storrs, CT (US)

(72) Inventors: Fotios Papadimitrakopoulos, West Hartford, CT (US); Antonio Costa, Hartford, CT (US); Faquir Jain, Storrs, CT (US); Santhisagar Vaddiraju, Plymouth, MN (US)

(21) Appl. No.: 15/290,468

(22) Filed: Oct. 11, 2016

Related U.S. Application Data
(63) Continuation-in-part of application No. 14/220,878, filed on Mar. 20, 2014.
(60) Provisional application No. 62/239,597, filed on Oct. 9, 2015.

(51) Int. Cl. A61B 17/34; A61B 5/145; A61M 25/06; A61M 25/01; A61M 5/315; A61B 5/06; A61B 90/13

(52) U.S. Cl. CPC A61B 17/3468; A61B 5/062; A61B 5/14503; A61B 17/3403; A61B 90/13; A61M 25/0127; A61M 5/315; A61M 25/0662; A61B 2034/2051

(57) ABSTRACT

A device and method are outlined for the manual or automated insertion and extraction of a miniaturized implantable biosensor underneath the skin. System comprises injection and extraction module that is in operable communication with a positioning and tracking module, microprocessor and data acquisition units. The positioning and tracking module utilizes light- or magnetic field-sensing arrays to provide spatial (x, y) position, depth (z) and rotational (□) state of the miniaturized implant. This is fed to the injection and extraction module that lines up a catheter. For extraction, the catheter is actively guided using sensing arrays to extract the biosensor. This system has also provisions to excise fibrosis tissue around the implant. This tool is operated in a manual or automatic mode to facilitate pain-free injection and extraction of a miniaturized biosensor with minimal trauma.

### CROSS REFERENCE TO RELATED APPLICATIONS

This application is a continuation-in-part of U.S. application Ser. No. 14/220,878, filed Mar. 20, 2014 and claims benefit of priority of the filing date of U.S. Provisional Application No. 62/239,597, filed Oct. 9, 2015, the contents of both of which are incorporated herein by referenced in their entireties.

### BACKGROUND OF THE INVENTION

Fully implantable biosensors for use in medical applications have significant promise in diagnosing and managing human diseases. A biosensor can be defined as any device that detects a chemical or physical change, converts that signal into an electrical or chemical signal and transmits the response to a secondary device (also referred to as a proximity communicator). An implantable biosensor can be implanted within the subcutaneous tissue space as well as within the layers of skin, intramuscularly or within the vasculature. Implanting the biosensor into these locations permits the sensing of analytes (e.g. glucose, lactate, molecular oxygen, glycerol, glutamate, hydrogen peroxide, etc.) for both discrete and/or continuous monitoring. An implantable biosensor requires energy to perform tasks and this energy requirement can be fulfilled by using a built-in battery, biofuel cells, photovoltaic cells, radiofrequency (RF) coils (i.e. RF powering), or by interacting with various forms of electromagnetic radiation (i.e. fluorescence, phosphorescence, Raman, etc.).

A biosensing platform typically comprises of two main systems: an implantable biosensor and an associated proximity communicator (e.g. a watch-like device worn on the wrist). In order for the biosensing platform to function, i.e. to operate and provide analyte information (e.g. glucose concentration), a communication link must be established and maintained between both systems. Such communication can be optical, near-infrared, or RF. The invention described herein outlines the automated insertion and extraction of a miniaturized implanted biosensor. This invention primarily relates to implantable biosensors that use optical powering (i.e. photovoltaic cells) and communication, although with certain provisions it can be extended to other powering and communication protocols.

Briefly, the optical powering and communication between the proximity communicator and biosensor is a two-way process that typically requires line-of-sight alignment between various components of the miniaturized implanted biosensor and its proximity communicator. Operation of the implant requires electromagnetic radiation source (e.g. a light-emitting diode or laser) located on the proximity communicator to provide sufficient radiant energy to power the biosensor. This light must pass through tissue to reach the photovoltaic cell(s) of the biosensor. Upon the light reaching the photovoltaic cell(s), the photovoltaic cell(s) convert such light into electricity that can be used to power the integrated circuitry on the biosensor. Here it is important to stress that the surrounding tissue causes this light to scatter that results in a reduction in the intensity of radiation reaching the photovoltaic cell(s). Such light scattering is proportional to both the depth of the skin that the light needs to travel through as well as to the 1/λ⁴ of its wavelength (λ). For optical wavelengths (i.e. red), if the implantation depth of the biosensor is too high, the amount of optical energy delivered is insufficient for proper operation of the biosensor. This necessitate that the implantation depth is accurately controlled in order to ensure that adequate light reaches the photovoltaic cells of the biosensor. The second process relates to the biosensor that upon powering, it is capable to transmit out to the proximity communicator electromagnetic radiation signals via its on-board source(s) (e.g. LEDs or lasers) operating at a different wavelength from that of the powering source. The proximity communicator further includes photodetectors that can detect such radiation with the help of adequate circuitry and a processor to convert such signals into an analyte concentration.

The implantation process of such a miniaturized biosensors can be accomplished by injection through a conventional, medical-grade needle/syringe. During this injection/implantation process, two problems can arise: (i) the strong light scattering nature of the tissue can impede a trained individual (e.g. a medical doctor or nurse) to insert the biosensor in its proper location and more importantly at the proper depth underneath the skin in order for the biosensor to receive adequate amount of light; and (ii) the biosensor can rotate, which reduces the effective absorption area of its photovoltaics cells as well as misaligns its on-board LEDs. Upon rotation, the proximity communicator is impeded from optically powering and communicating with the biosensor, which can ultimately result in the biosensing platform to be inoperable. Therefore, during implantation, the biosensor must be tracked and properly aligned such that post-implantation, the biosensor and proximity communicator have their powering and communication modules within a line-of-sight of each other.

For non-biodegradable implants, following the completion (or before) of their useful lifetime, the biosensor needs to be extracted. Biosensor extraction is typically much more challenging than insertion and requires significant more skill from a trained individual (e.g. a medical doctor or nurse) to explant it. This stems from the fact that: (i) the miniaturized implant is difficult to be visually located or felt; (ii) the brittleness of the implant can cause it to fracture upon handling with typical tweezers, forceps, etc.; and (iii) the surrounding tissue can grow around the implant (typically referred as fibrosis) imposing difficulties in the extraction process. The latter necessitates that the surrounding tissue is excised together with the implant. Typically a trained professional needs to perform surgery to carefully detach the implant from the surrounding tissues, apply particular care not to fracture the biosensor with the potential of leaving fragments behind, and close the wound via suture or surgical glue.

In order from impeding the surrounding tissue to fibrose around the implant, a variety of methods have been developed over the years. A particular method that applies for rigid (silicon- or glass-based implants) surrounds the sensor with a biocompatible coating that affords the slow and steady release of anti-inflammatory agents (e.g. dexamethasone). Such methodology has been proven effective in suppressing fibrosis so long that the anti-inflammatory agent is present in the surrounding tissue at sufficient concentrations. Furthermore, it is important to stress that fibrosis gradually sets in upon exhaustion of such anti-inflammatory agent. Such an exhaustion typically defines both the useful lifetime of the bio sensor and the optimum window for biosensor extraction before fibrosis sets in. Performing implant extraction within such a window can significantly simplify the extraction process and facilitate an automated process that eliminates the need for surgical operation.

### SUMMARY OF THE INVENTION

This invention describes an apparatus and associated methods for the automated insertion and extraction of a miniaturized, needle-implantable biosensor. Such device (apparatus) and methodology ensures that the miniaturized biosensor is implanted at the desired spatial (x, y) position, at the desired depth (z) and with the appropriate orientation (φ) (or otherwise termed alignment) with respect to its proximity communicator to ensure an optimized optical powering and communication protocols. In addition, the similar apparatus and method can be used to locate (or otherwise termed “track”) and explant the implantable biosensor after its useful lifetime. Such device facilitates both the extraction needle and implanted biosensor to adopt the right alignment so that explantation can take place solely with a needle, herein termed insertion and extraction catheter. Such automated insertion and extraction tool, which can be also operated manually, requires minimal user intervention and it is intended to minimize cost and facilitate pain-free injection (i.e. implantation) and extraction (i.e. explantation) procedures with minimal trauma.

As described above, maintaining the proper alignment of the biosensor during the insertion process is critical for optimal sensor function. This necessitates that the biosensor does not rotate during insertion. This ensures that the on-board photovoltaic (PV) cell(s) of the implant are facing upwards towards the skin (i.e. the normal of the PV cell is also normal to the skin) to warrant that the light from the proximity communicator has the shortest and most direct path to the photovoltaic cell(s) of the biosensor, in order to minimize light attenuation due to scattering.

A number of three-dimensional (3D) imaging methods can be used to locate the exact spatial (x, y) position and depth (z) of the implanted biosensor. While this invention is compatible with the majority of 3D imaging techniques, the following two methods are particularly suited since they incorporate similar imaging methods used in the proximity communicator to track the implant on the fly (i.e. during exercise with the proximity communicator loosely bound to the arm, allowing the skin to breath): A) The first approach is based on the biosensor comprising permanent magnets, electromagnets or magnetically susceptible materials that create or interact with a magnetic field around the implant. B) The second approach is based on the biosensor comprising of multiple electromagnetic radiation sources (i.e. LEDs or lasers) that generate a well-defined light-emission pattern around the implant. Both approaches incorporate two-dimensional array of sensors (i.e. magnetic field detecting sensors and photodetectors for A and B, respectively) that are incorporated into the proximity communicator device. In this invention, the use of such magnetic field detecting sensors and photodetectors are appropriately adapted for the described injection and extraction apparatus. These magnetic field detecting sensors and photodetectors arrays utilize the amplitude response of their individual sensors to generate a three-dimensional mapping (in terms of x, y, and z coordinates of either ends of the rod-like implant as well as its precise biosensor alignment (defined by the rotation angle (φ) (with respect to the normal of the skin) and tilt angle (θ) (with respect to the skin surface). This mapping information is used to provide key information on the precise position of the implant that is fed to a computer algorithm to automatically guide the extracting needle to the exact position of the implant. Moreover, the aforementioned imaging arrays also provide active feedback on how the injection (implantation) and extraction (explantation) processes as well as troubleshoot and provide corrective action in the case of an abnormal response is detected. The system further enables the tracking of the biosensor during and upon implantation in a medium (e.g. subcutaneous tissue) that is an obstruction to human vision. In addition, the invention satisfies the requirement that the implant and the proximity communicator must be properly aligned to initiate operation immediately upon implantation.

### BRIEF DESCRIPTION OF THE DRAWINGS

The foregoing and other features and advantages of the present invention will be more fully understood from the following detailed description of illustrative embodiments, taken in conjunction with the accompanying figures in which like elements are numbered alike:

FIG. 1. Block diagram layout of the automated insertion and extraction apparatus with respect to the user extremity.

FIG. 2. Main components of the automated insertion and extraction apparatus.

FIG. 3. Magnetic-assisted alignment of the automated insertion and extraction apparatus with respect to the miniaturized implant.

FIG. 4. Light-assisted alignment of the automated insertion and extraction apparatus with respect to the miniaturized biosensor implant.

FIG. 5. Magnet-outfitted plunger-tip-alignment-element ensures proper implant orientation during insertion.

FIG. 6. Insertion catheter equipped with a forward-oriented, spring-loaded, hinge-activated flap, which is interfaced with a flap-release rod.

FIG. 7. Insertion catheter equipped with magnet-assisted implant captures and boring catheter.

FIG. 8. Insertion catheter equipped with magnet-assisted implant captures, boring catheter and hinge-activated flap.

FIG. 9. Insertion catheter equipped with a backward-oriented hinge-activated flap.

FIG. 10. Method flowchart for the automated insertion of a miniaturized implantable biosensor.

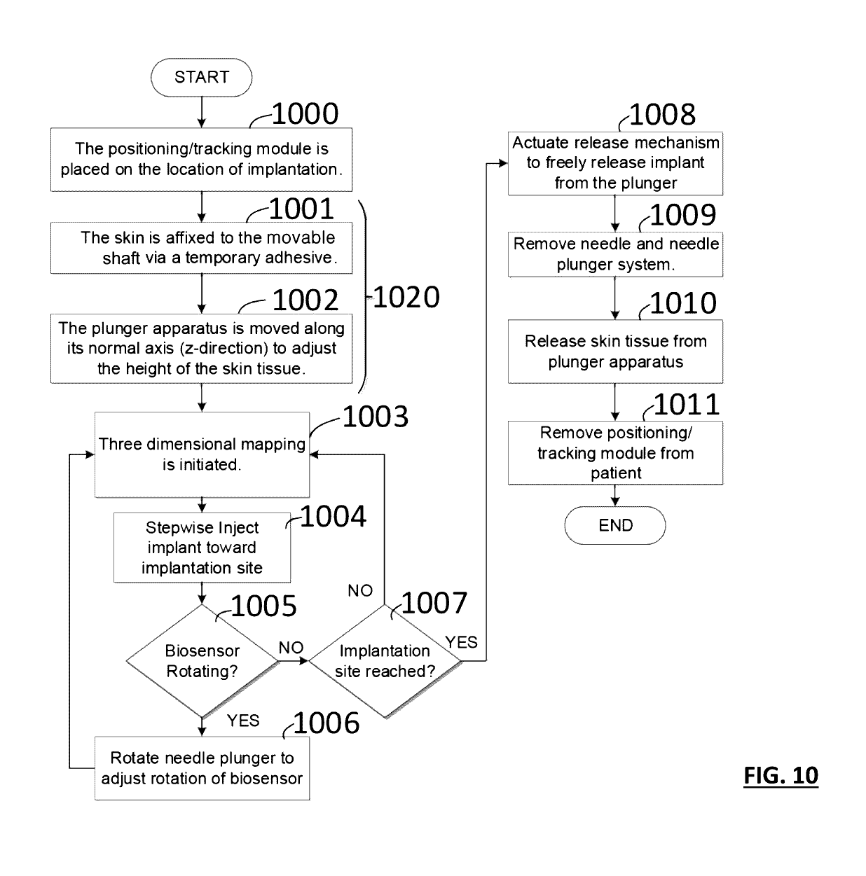

FIG. 11. Method flowchart for the automated extraction of a miniaturized implantable biosensor.

### DETAILED DESCRIPTION OF THE INVENTION

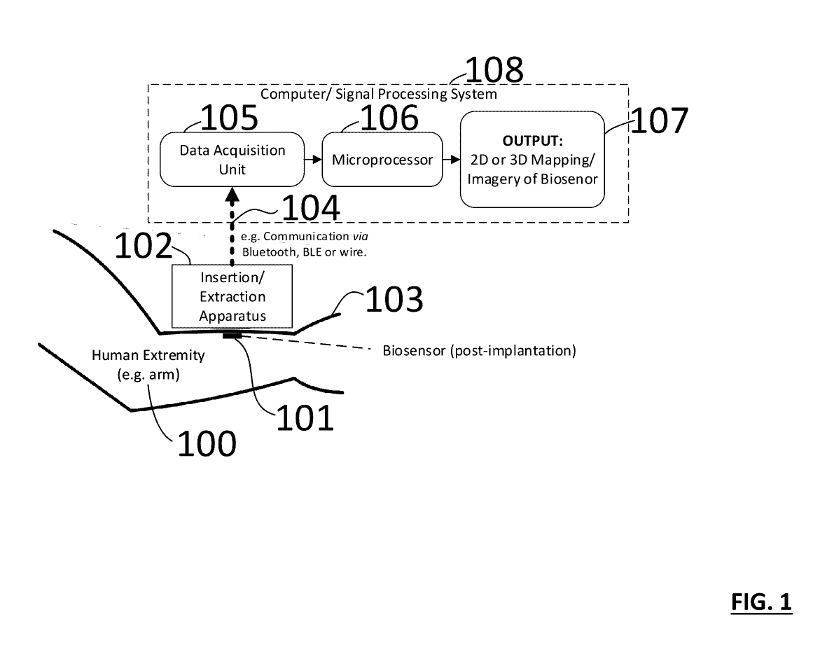

Description of the Injection/Extraction System—FIG. 1 describes the automated insertion and extraction apparatus in terms of block diagram. The insertion and extraction module 102 communicates via Bluetooth or wires 104 to a computer/signal processing system 108. The computer/signal processing system 108 further comprises from a data acquisition unit 105, a microprocessor 106 and associated computer algorithms that generate a detailed two-dimensional (2D) and three-dimensional (3D) mapping/imagery 107 of the implantable biosensor 101 with respect to the skin 103. In one example, the insertion apparatus is used to inject a miniaturized biosensor 101 into a human extremity 100 in the vicinity of the skin 103 (e.g. in the subcutaneous space). The above example is directed toward humans; however, the invention can also apply to animals and plants.

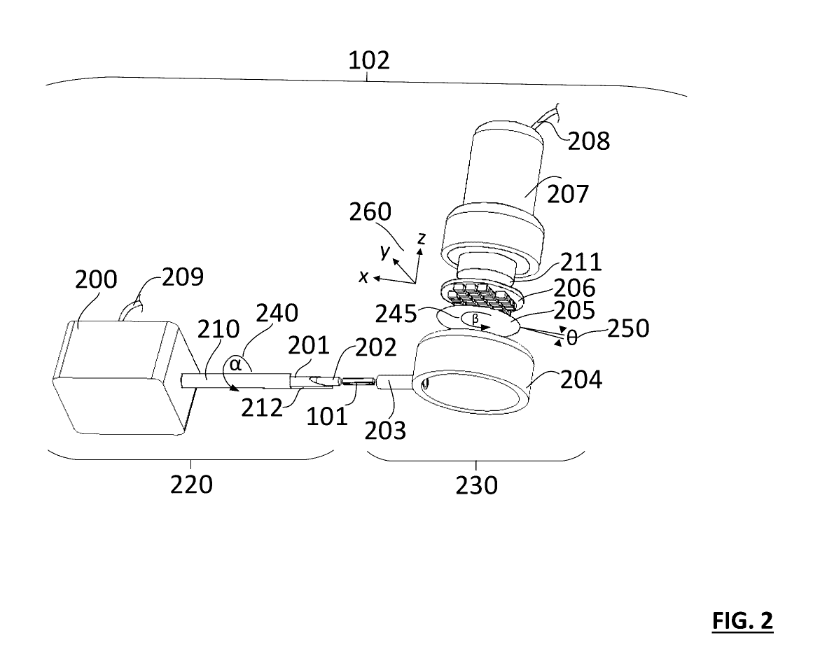

The miniaturized implantable biosensor 101 is small enough in two of its dimensions (i.e. height and width) to fit through a hypodermic needle (FIG. 2). A typical size of the miniaturized implantable biosensor is 1×1×5 mm, although its height and width can vary from few nanometers to few millimeters and its length can vary from few nanometers to tens of millimeters (e.g. 50 mm). The miniaturized implantable biosensor 101 can be composed of multiple materials (i.e. semiconductors, metals, magnets, magnetic susceptible compound, insulators, biomaterials (i.e. enzymes, proteins, DNA, sugars, etc.) polymers, bio-polymers, drugs, tissue response modifying agents, nanomaterials (i.e. carbon nanotubes, graphene-based materials, semiconductor nanoparticles, single chain polymer nanoparticles, nano-rods, nano-platelets, etc.), sub-devices (i.e. electronic, optoelectronic, electro-optic, photonic, electrochemical, magnetic structures and circuits) and a variety of specialty structures that assist in controlling permeability, sustained delivery of drugs and tissue response modifying agents, increasing surface area, prevent or facilitate swelling, as well as provide stability for enzymes and various biologics against freezing and drying conditions.

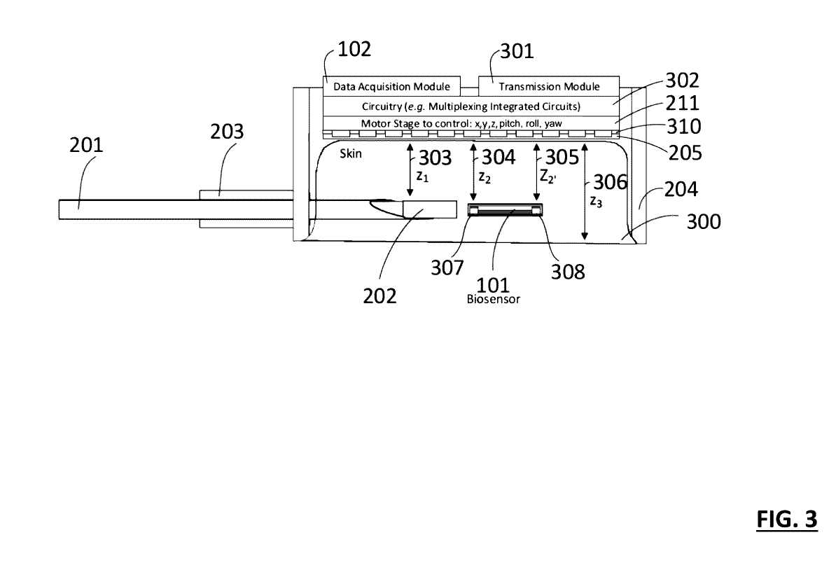

As a first example, the miniaturized biosensor implant is equipped with magnetic or magnetic susceptible materials (i.e. permanent magnets, electromagnets, or specialized structures (such as coils, coils wrapped around rods or other two- and three-dimensional structures made from non-magnetic or magnetic susceptible materials) that in the presence of an external magnetic field, they interact with the external field and slightly alter it). One such embodiment involves equipping the miniaturized biosensor implant 101 with two small magnets 307 and 308 at its either ends (FIG. 3.) A typical size of these magnets is 0.75×0.75×0.75 mm, although their size can vary from few nanometers to few millimeters. These magnets are placed within the miniaturized biosensor implant in a predetermined North (N)-South (S) polarity (N/S). In another embodiment, the permanent magnets are replaced with electromagnets that are powered by the miniaturized implant. Such magnets or electromagnets generate a well-defined magnetic field, which can be assessed with the help of magnetic-field imaging devices. Hall effect sensors and giant magneto resistance (GMR) devices are appropriately suited to detect both strength and polarity of magnetic-fields. By arraying and multiplexing such magnetic field sensors, one can extract the spatial (x and y) location of an implanted magnet, as well as its proximal depth (z) and rotation angle of is N/S polarity (co) with respect to the normal of the skin (FIG. 3). In yet another embodiment the permanent magnets or electromagnets 307 and 308 are changed with specialized structures comprised of coils, coils wrapped around rods or other two- and three-dimensional structures made from non-magnetic, minimally magnetic, or magnetic susceptible materials like traditional metals, organic and graphitic conductors or spin glass. Such specialized magnetic susceptible structures provide sufficient interaction with an external magnetic field, to also impart localization means with the aforementioned arrays of magnetic-field imaging devices.

FIG. 2 illustrates the main components of the automated insertion and extraction device (apparatus) 102. This device may include two modules: an injection and extraction module 220 and a positioning and tracking module 230.

The injection and extraction module 220 includes a motorized unit 200 equipped with various stages that provide linear translation and rotary motion (angular rotation) to various elements, along with force and motion sensors and associated circuitry. The motorized unit 200 is also equipped with a communication link 209 and the insertion and extraction catheter 201, typically a regular or appropriate modified hypodermic needle. Within the insertion and extraction catheter 201 resides the catheter plunger 202, and its flap-release rod 212. Outside of the insertion and extraction catheter 201, resides a concentric boring catheter 210, (typically composed of a square face hypodermic needle with sharp edge and optional microscopic teeth). The motorized unit 200 provides independent linear movement to the insertion and extraction catheter 201, plunger 202, flap-release rod 212, and the boring catheter 210. Such movement can be performed concurrently for all for four components (i.e. typically during skin insertion), or independent from each other. In addition, aside from the linear translation, angular rotation 240 is also available for the boring catheter 210, plunger 202 and the combined insertion and extraction catheter 201 with its flap-release rod 212. Moreover, the boring catheter 210 it can further rotate at variable speeds and in both directions in order to assist with boring (herein defined as circular excising process around the implant 101).

The positioning/tracking module 230 function is to lift up the skin, localize the miniaturizeds biosensor implant 101, and actively guide both insertion and extraction process (in both manual and/or automatic mode). The positioning and tracking module 230 includes an injection port 203, a housing unit 204, an adhesive layer 205, an array of sensors 206 with associated electrical circuitry, a moveable shaft 207, and a communication link 208. Within the moveable shaft resides a motorized stage 211, that provides movement along x, y, and z axes 260, as well as in a rotary (β) 245 or pitch, tilt (θ) 250 or yaw and roll fashion. This stage is affixed and manipulates the exact position of the array of sensors 206 and its adhesive layer 205. The purpose of the injection port is to direct the insertion and extraction catheter 201 into the tissue space (e.g. subcutaneous tissue), as shown in FIG. 3. By lowering the moveable shaft 207, the adhesive layer 205 comes in contact with the skin 300 and affixes it to the array of sensors 206. Subsequently, the moveable shaft is raised to an appropriate height 306 (ca. 3 mm) with respect to the rigid rim of the housing unit 204. Such action lifts up the skin 300 in a “π” shape form (FIG. 3).

In one embodiment the positioning and tracking module for the miniaturized implant 101 is based on magnetic field detecting sensor array 310. This array enables the determination of the x, y, z and rotation angle (φ) coordinates (i.e. (x₁, y₁, z₁, φ₁) and (x_{1′}, y_{1′}, z_{1′}, φ_{1′})) of the magnets (307 and 308) at either ends of the implant, respectively. These eight coordinates enable the magnetic field detecting sensor array 310 to ascertain the precise depth and rotation of the implant. From the difference between z₁ 304 to z_{1′} 305, one can also determine the tilt angle (θ) 250 of the implant with respect to the skin surface. Such information is fed in the motorized stage 211 in order to perform the necessary movements to align the implant on the same axis of the insertion and extraction catheter 201. Moreover the rotation information φ₁ and φ_{1′} (which is the case of antiparallel magnet 307 and 308 polarizations should be φ₁ and φ_{1′}=φ₁180°) is also fed in the microprocessor 106 to appropriately adjust the rotation of the insertion and extraction catheter 201 to match the orientation of the implant 101.

In another embodiment, the magnets 307 and 308 are replaced with electromagnets. In such case, the 310 array should be composed of two arrays in coplanar of stacked configuration: (i) an array of magnetic field detecting sensors; and (ii) an array of LEDs or RF power sources to power the implant. In the case of light powering, the array of LEDs is placed directly over the adhesive layer 205 (which is transparent to light).

Another embodiment exchanges the magnets 307 and 308 or electromagnets with specialized structures comprised of coils, coils wrapped around rods or other two- and three-dimensional structures made from non-magnetic, minimally magnetic, or magnetic susceptible materials. Since these materials do not generate a magnetic field by themselves, they require an external magnetic field that in their presence, the said magnetic field is slightly altered. Such magnetic-field generating devices can be located within the housing unit 204, or behind the magnetic field detecting sensors array 310.

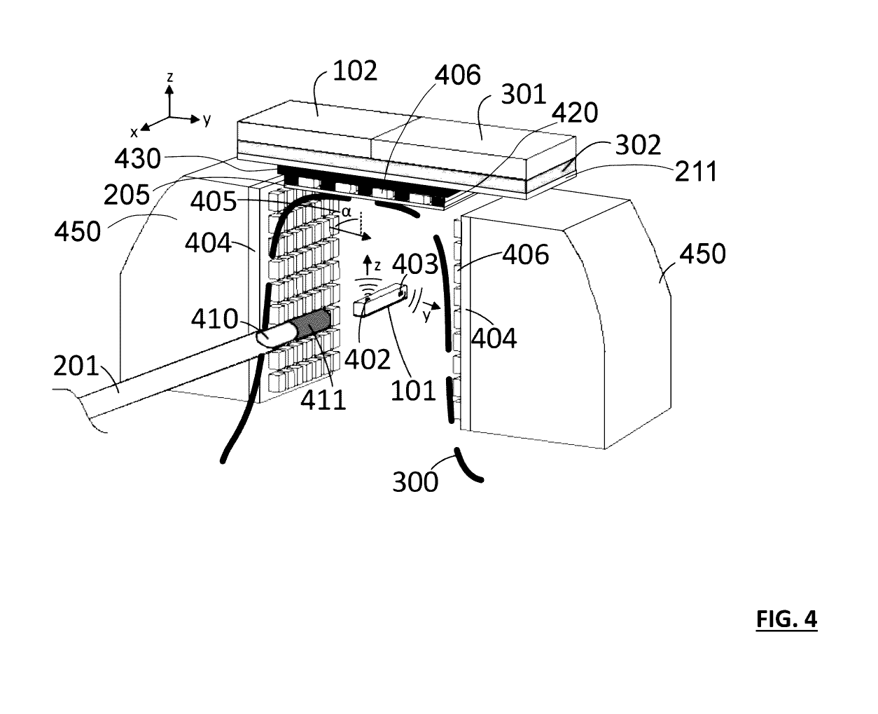

In yet another embodiment the magnets, electromagnets and magnetic susceptible materials at the tips of the implant can be exchanged with light-reporting devices (i.e. LED or lasers). FIG. 4 illustrates the light-assisted alignment of the automated insertion/extraction apparatus with respect to the miniaturized implant. Here the skin is first affixed to the temporary adhesive layer 205 and then it is lifted up by the moveable shaft 207, as shown in FIG. 2. This provides the clearance for two pinching shafts 450 to move towards each other and pinch the skin 300 as shown in FIG. 4. Such configuration confines the miniaturized implantable biosensor 101 at the center of pinched skin. At the opposing ends of each pinching shaft 450 reside an array of photodetectors (PD) 404. This embodiment employs the use of an array photodetectors (PD) 404 to located on the exact position of the miniaturized implant 101. These photodetectors 406 receive light from two or more light sources (e.g. LEDs or lasers) 402 and 403 located on the miniaturized implantable biosensor that are oriented at a defined angle with each other (e.g. at 90°). In order to activate the implants' light sources 402 and 403, one has to power the implantable biosensor by light or RF. Such powering can be ensured by one or more proximal LEDs 420 located on the bottom of the motorized stage 211. In order to ensure homogeneous light powering, an array of powering LEDs can be co-located within the array of PDs 430. By simultaneous illuminating all light sources in the LED/PD array 430, the implanted biosensor device 101 is sufficiently powered and its two light sources 402 and 403 are activated. If more light power is needed, the two side PD arrays 404 can be also exchanged with LED/PD arrays 430. By simultaneously scanning each photodetector in the PD array, the amplitude of the emitted light (i.e. intensity) can be established at each photodetector. This result is an intensity map from the three photodetector arrays that can then be used to determine the spatial (x, y), depth (z) and rotational (□) position of a miniaturized implant within a highly scattering tissue. This enables the determination of the exact location of the miniaturized biosensor that with the help of a 6-axis (x, y, z, pitch, roll, yaw) motorized stage 211 it can interactively align the insertion/extraction catheter 201 along the long axis of the implant.

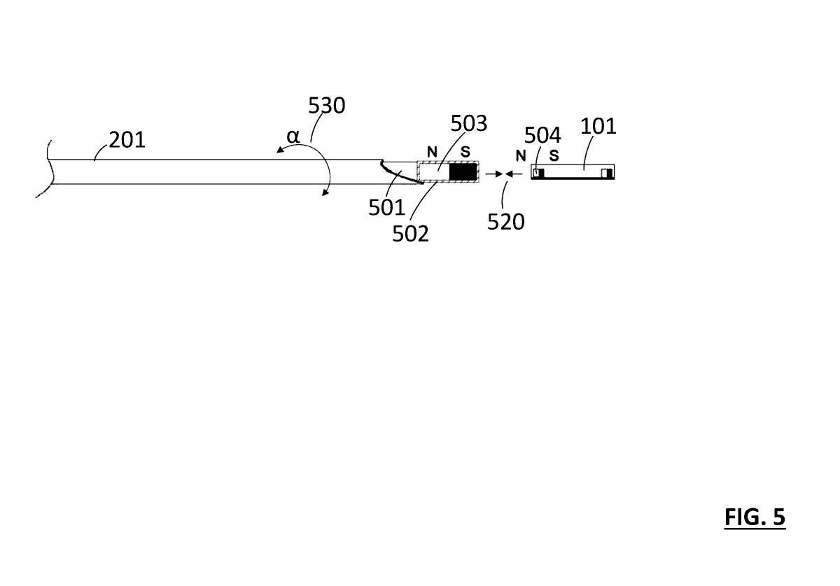

In order for the aforementioned embodiments to perform optimally, they also necessitate appropriate modifications on the insertion and extraction catheter 201 and its plunger 202. One of the prime requirements for optically powered biosensors is to be implanted with their photovoltaic (PV) cells facing upwards towards the skin (i.e. the normal of the PV cell is also normal to the skin). Consequently, the automated injection/extraction device 102 described herein, must also includes provisions to ensure that the implantable biosensor is properly aligned during implantation. FIG. 5 illustrates a representative example in conjunction with the magnetic-assisted tracking embodiments described above. Here, the plunger 501 that resides within the insertion and extraction catheter 201 is equipped with a magnetic plunger tip 503 (also defined as plunger-tip-alignment-element), which is optionally coated with biocompatible coating 502 to improve its bio-compatibility. Such magnetic plunger tip 503 is designed to have sufficient magnetic strength 520 to be temporarily affixed with the miniaturized biosensor 101. In this manner, the polarity at the magnetic plunger tip 503 matches that of the magnet 504 located at the selected tip of the miniaturized biosensor 101. The magnetic plunger tip can be a permanent magnet or electromagnet. Upon interrupting the current that runs though the electromagnet, its magnetic force is substantially reduced, which makes it a better candidate for implant insertion. Here, it is important to recognize that electromagnet do not to demagnetize completely, which means that a finite attractive force 520 will persist even with no current flowing through the electromagnet. One potential venue to actively separate the magnetic plunger tip 503 from the implant 101 is to bias the electromagnet with an opposite polarity current. This will reverse the electromagnet polarity and push away the miniaturized biosensor. Here it is important to stress that significant care must be applied to the magnitude of the reverse current. Such current must be kept sufficiently low to ensure that the opposite polarity of the magnetic plunger tip is not strong enough to cause the miniaturized biosensor to flip or jump up on tip in order to match the reverse polarity.

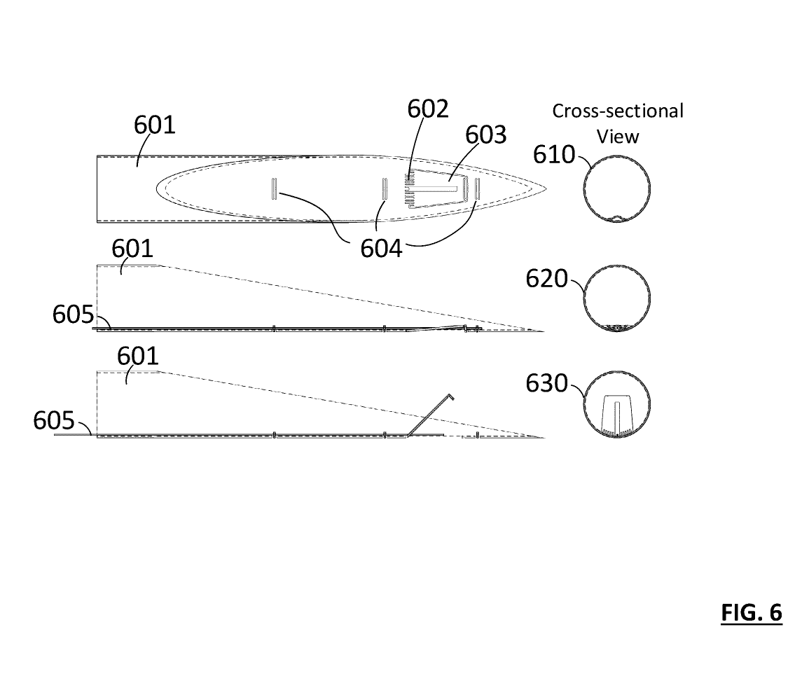

Another venue to separate the magnetic plunger tip 503 from the implant 101 is to introduce a physical barrier in between these two objects. Such physical barrier can be a spring-loaded, hinge-actuated flap such as that shown in FIG. 6. Here the insertion catheter 601 is appropriately modified via micromachining, or other methods, to incorporate a hinge 602, a spring-loaded movable flap 603, and rod guides 604. Such rod guides are clearly viewed at cross-section 610, which allow a rod 605 (defined as flap-release rod) to be guided through. The movable flap 603 is first deformed upwards along the hinge cuts 602. Such deformation acts as a spring. The flap is then pushed downwards and locked 620 by the movable rod 605. In this action, the flap gets spring loaded. Once the release-rod slides back, the stored mechanical stress on the hinge moves the flap upwards 630. Such flap can physically prevent the biosensor from following the retracting plunger and re-entering the insertion catheter. Such catheter 601 can be made from micro-machined stainless steel rod. The catheter 601 is discarded after each use. The same applies to the implantation and extraction catheter 201, plunger 202, plunger tip 503, and boring catheter 210, and flap-release rod 212.

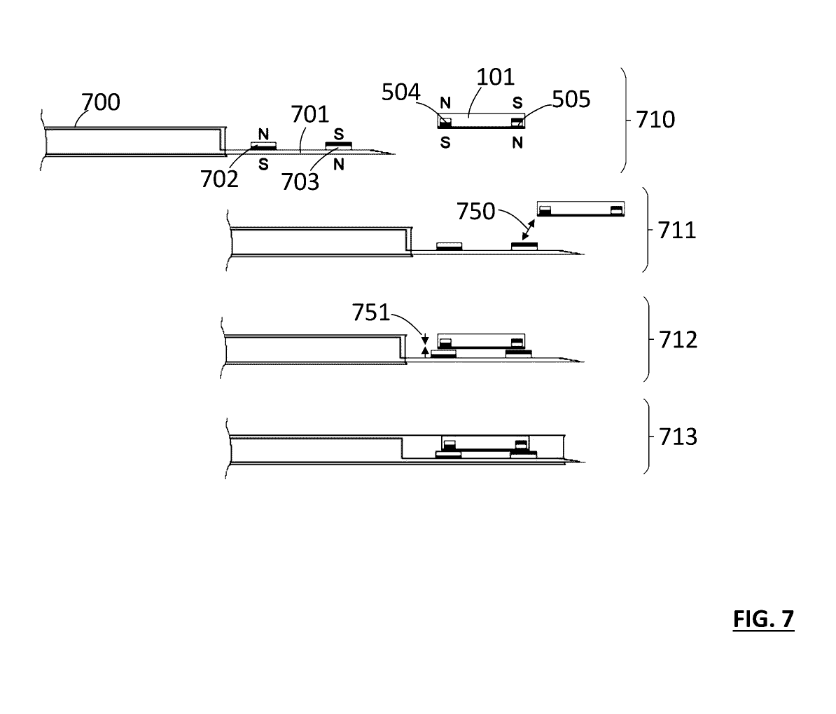

FIG. 7 illustrates another embodiment for the insertion and extraction catheter that is applicable to magnetic-assisted tracking and automated extraction of magnet-equipped miniaturized implantable biosensors. A foreign body capsule (also referred as fibrosis or fibrosis capsule) that might build around the implant typically complicates biosensor removal at the end of its useful lifetime. The magnitude of such capsule will greatly impede implant extraction, which necessitates to equip the automated insertion and extraction device with additional capabilities.

First is described the implant removal in the case where the foreign body capsule is minimal to virtually absent. Here, the tip of the extraction catheter 701 is equipped with two magnets (702 and 703), with polarities matching that of the magnets (504 and 505) on the miniaturized implantable biosensor. Upon skin insertion, the magnetic-field sensor array 310 tracks both catheter (702 and 703) and implant (504 and 505) magnets. The position of all four magnets is fed to the microprocessor to adjust: (i) the linear-translation and angular rotation of the extraction catheter 701; and (ii) the 6-axis (x, y, z, pitch, roll, yaw) motorized stage 211 that controls the position of the implant. This permits the catheter to be guided in proper alignment to capture the miniaturized implanted biosensor. Such capture is facilitated by the magnetic attraction 751 of the biosensor magnets (504 and 505) to the extraction catheter magnets 702 and 703. This action can be verified by the magnetic-field sensor array 310, by producing a noticeable change upon the magnets latching on with their mates. Such magnetic attraction 751 might be sufficient to enable implant extraction upon withdrawal of the catheter. This can be augmented by the attraction force 520 of a plunger equipped with a magnetic tip.

In the case where the fibrose capsule is substantial, the aforementioned magnetic forces (751 and 520) will be incapable to dislodge the implant. FIG. 7 illustrates the use of a larger-diameter boring catheter 700 placed around the extraction catheter. Such boring catheter can be rotated and translated at an appropriate speed so that it slowly cuts the fibrose tissue around the implant. Here, magnetic attraction provided to the implant by the extraction catheter 751 and plunger 520 (not shown in FIG. 7) play an important role in implant localization while slowly cutting the fibrose tissue around it 713. Such action is guided by the magnetic-field sensor array 310 that tracks both catheter (702 and 703) and implant (504 and 505) magnets to ensure that they remain latched on. In the case that the rotation of the boring catheter 700 tends to dislodge the implant from the catheter, the boring catheter rotation can be reversed. In another embodiment, the boring catheter can advance using a back and forth rotation.

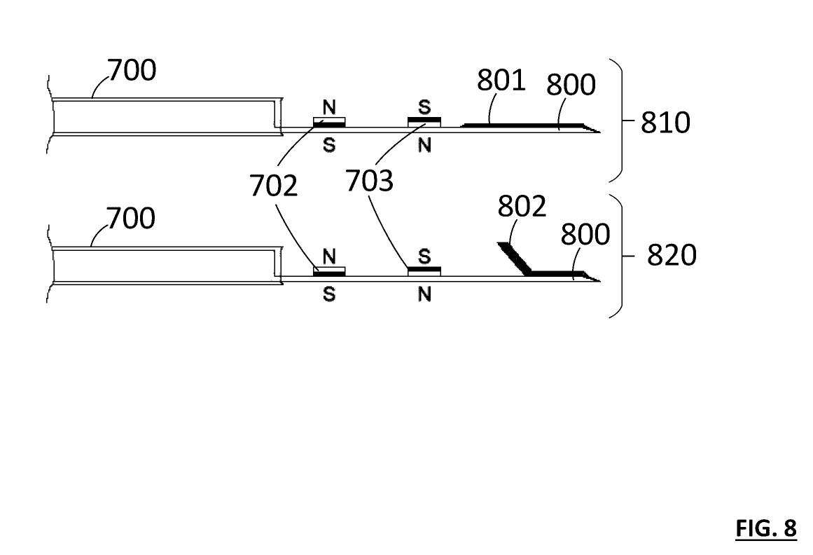

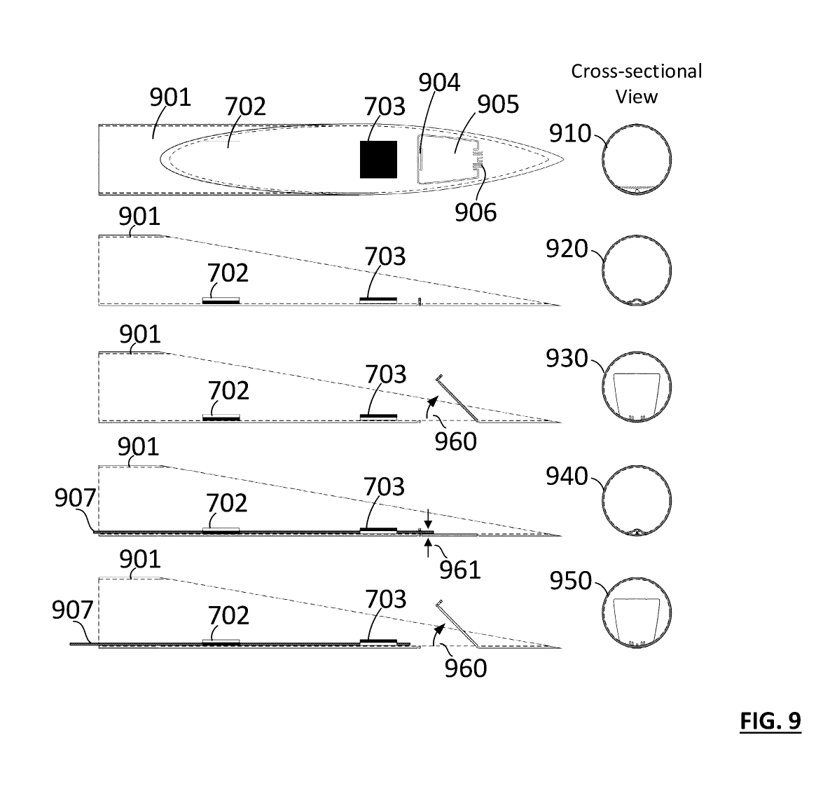

Following successful boring around the miniaturized biosensor implant 713, one might also need to sever the remaining tissue behind the implant to truly release it. Such action can be performed with a movable excision shaft situated on the tip 800 of the extraction catheter (FIG. 8). In one embodiment, the extraction catheter tip transforms its shape from 801 to 802. Mechanical-, electrical- or temperature-induced actuation can stimulate such transformation. FIG. 9 depicts an exemplary method to realize such movable excision shaft situated on the tip 800 of the extraction catheter. Here the extraction catheter 901 is appropriately modified through micromachining or other methods to incorporate a hinge 906, a spring-loaded movable flap 905, and a latch 904. Through appropriately shaped magnets (702 and 703), the rod 907 is guided under them. The movable flap 905 is first deformed upwards 960 along the hinge cuts 906 and then pushed downwards and locked in place 961 at the latch 904, with the rod 907 (also defined as flap-release rod). Once the rod 907 slides backwards, the stored mechanical stress on the hinge 906 moves the flap upwards 960. Electrical discharge micromachining (EDM) can be used in such manner to also produce a sharp edge (i.e. 30°) of the flap along the 904 latch. Such a sharp edge can greatly facilitate tissue severance using the movable flap 905, in the case where the boring catheter is kept constant and the extraction catheter with is raised flap is withdrawn backwards.

Description of Method: Implantation of Biosensor using the Positioning and Tracking Module—Insertion of the biosensor begins with placing the positioning/tracking module onto the location of where the implant will be injected 1000. A temporary adhesive layer 205 is then activated (i.e. by pilling off a protective coating to expose the adhesive surface) and attach the skin 300 to the imaging array 205, which in turn is attached to the a moveable shaft 1001. The moveable shaft 207 is then moved along the normal axis of the skin (z-direction) to adjust the height of skin tissue pulled upward 1002. The above two steps 1001-1002 can be combined into the initialization of the positioning and tracking module 1020. The three dimensional mapping is then activated to visualize the biosensor as it is implanted and to track/align the implant during the injection process 1003. The implant is injected in a gradual manner so that appropriate time is provided to the 6-axis (x, y, z, pitch, roll, yaw) motorized stage to guide the insertion catheter tip to the appropriate depth 1004. During the injection, a YES/NO decision is made for if the biosensor has rotated 1005. Upon biosensor rotation, the needle plunger is rotated to re-align the biosensor into the pre-determined orientation. Subsequent a YES/NO decision is made for if the biosensor reached the desired implantation site 1007. A NO answer sends the process to restart at three-dimensional mapping 1003. Upon the biosensing reaching the implantation site, the release mechanism is actuated to release the biosensor from the plunger 1008. The insertion catheter (referred as needle) and plunger (referred as needle plunger) are then removed from the patient 1009. The skin tissue is then released from the piston apparatus 1010 and the positioning/tracking module is removed from the patient 1011.

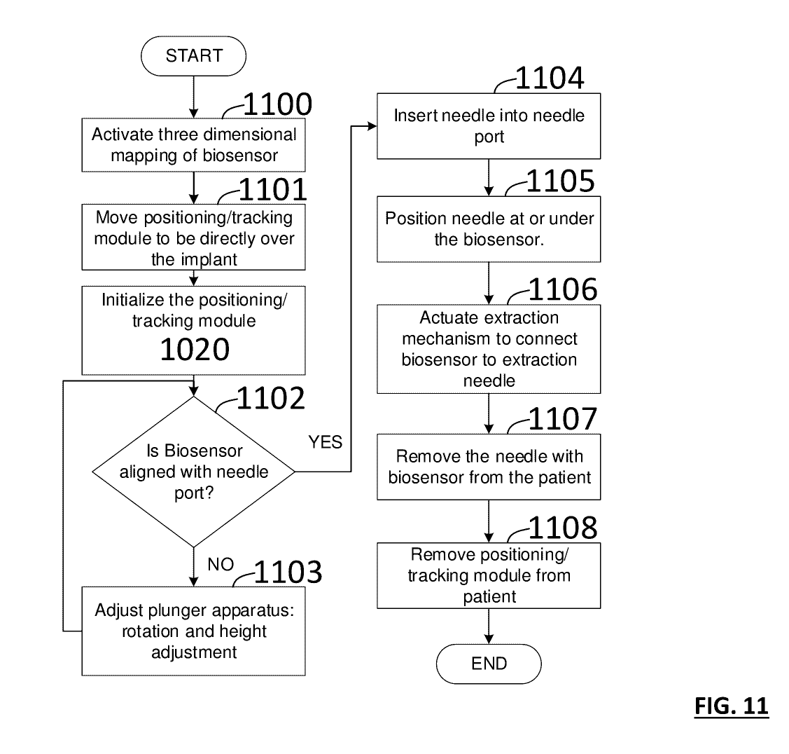

Description of Method: Extraction of Biosensor using the Positioning and Tracking Module—The extraction of the biosensor starts with the activation of the three-dimensional mapping of the biosensor 1100 to determine its exact spatial location and rotational state. The positioning and tracking module is moved to be directly over the implant and centered on the implant 1101. The positioning and tracking module is then initialized 1020 as described above. A YES/NO decision 1102 is performed to determine if the biosensor 101 is properly aligned with the needle port 203, i.e. both the height of the needle port and implant height are within a tolerable distances and the longitudinal axis of the implant is aligned with the longitudinal axis of the needle port 203. Upon the biosensor not being properly aligned, the 6-axis (x, y, z, pitch, roll, yaw) motorized stage 211 readjusts the skin height, while the extraction catheter 201 (referred as needle in FIG. 11) rotates appropriately to align to the rotational state of the implant 1103. Upon the biosensor 101 is aligned with the needle port 203, the extraction catheter is inserted into the needle port 1104. The extraction catheter is then actively guided under the biosensor 1105. Subsequently, the extraction mechanism is actuated 1106, which proceeds either by a simple withdraw of both extraction catheter 201 and plunger 202 or by excising the tissue around the implant with the boring catheter 210. Following that, the extraction catheter 201 (referred as needle), plunger 202, and boring catheter 210 are removed from the patient 1107. The positioning/tracking module is then also removed from the patient 1108.

This injection and extraction tool can be operated in a manual or automatic mode to facilitate pain-free injection and extraction of a miniaturized biosensor with minimal trauma. To eliminate pain topical anesthetic creams or sprays (containing i.e. lidocaine, prilocaine, benzocaine, etc.) can be applied onto the skin to provide local anesthesia. There creams should be applied slightly before the miniaturized biosensor insertion and extraction procedure, while a dermaroller or other microneedle-based devices have been applied to break the continuity of the skin and facilitate absorbance of the local anesthetic. Insertion and extraction of the miniaturized biosensor should be performed on cleaned and disinfected skin with all the parts of the described device properly sterilized. Similarly, following of miniaturized biosensor insertion and extraction, local application of a scar-treatment and scar-prevention creams can be extremely effective in minimizing any catheter-induced scar, regenerate the skin, facilitate healing, and reduce any swelling and redness.

There are many embodiments that can be envisioned by users skilled in the art of the invention described here. For example, there are multiple schemes to image the skin and ascertain the exact location of a miniaturized implant. Lifting up the skin in a “π” shape form also lifts up the miniaturized implant and aligns it appropriately for the incoming extraction catheter. Fine adjustment by a multi-axes stage fine-tunes the alignment of the incoming catheter with the miniaturized implant for its capture and extraction. This method is also applicable for the automated injection of miniaturized implant. In the case that fibrous tissue impedes such extraction, a boring/cutting catheter is also used to first excise the tissue around the implant before extracting it.

In accordance with the present invention, it should be appreciated that the invention as disclosed herein may be implemented as desired via any devices suitable to the desired end purpose, such as digital devices, analog devices and/or a combination of digital and analog devices. Additionally, although the invention is disclosed herein with regards to one device, it is contemplated to be within the scope of the invention that a plurality of devices may be connected together (or integrated together) to achieve the same or similar results.

In accordance with the present invention, the processing of the invention may be implemented, wholly or partially, by a controller operating in response to a machine-readable computer program. In order to perform the prescribed functions and desired processing, as well as the computations therefore (e.g. execution control algorithm(s), the control processes prescribed herein, and the like), the controller may include, but not be limited to, a processor(s), computer(s), memory, storage, register(s), timing, interrupt(s), communication interface(s), and input/output signal interface(s), as well as combination comprising at least one of the foregoing.

Moreover, the method of the present invention may be embodied in the form of a computer or controller implemented processes. The method of the invention may also be embodied in the form of computer program code containing instructions embodied in tangible media, such as floppy diskettes, CD-ROMs, hard drives, and/or any other computer-readable medium, wherein when the computer program code is loaded into and executed by a computer or controller, the computer or controller becomes an apparatus for practicing the invention. The invention can also be embodied in the form of computer program code, for example, whether stored in a storage medium, loaded into and/or executed by a computer or controller, or transmitted over some transmission medium, such as over electrical wiring or cabling, through fiber optics, or via electromagnetic radiation, wherein when the computer program code is loaded into and executed by a computer or a controller, the computer or controller becomes an apparatus for practicing the invention. When implemented on a general-purpose microprocessor the computer program code segments may configure the microprocessor to create specific logic circuits.

It should be appreciated that while the invention has been described with reference to an exemplary embodiment, it will be understood by those skilled in the art that various changes, omissions and/or additions may be made and equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims and/or information. Moreover, unless specifically stated any use of the terms first, second, etc. do not denote any order or importance, but rather the terms first, second, etc. are used to distinguish one element from another.

We claim:

1. A device for the insertion and extraction of a miniaturized biosensor implanted underneath the skin comprising of:
an insertion and extraction module comprised of a catheter, a plunger and a boring catheter that are interfaced with linear translation and rotary movement,
a positioning and tracking module comprised of an injection port, a housing unit, of a moveable shaft, a multi-axes motorized stage, an array of imaging devices, and a temporary adhesive layer,
a microprocessor-based signal processing module,
wherein said catheter is outfitted with a spring-loaded flap that is activated by a flap-release rod,
wherein within said catheter resides a said plunger and said flap-release rod,
wherein said plunger is outfitted with a specialized tip,
wherein said catheter resides within a concentric boring catheter,
wherein said array of imaging devices is in contact with the skin via a an adhesive layer,
wherein microprocessor-based signal processing module is comprised of a data acquisition unit, display, and signal processing algorithms for real time imaging the location of an implanted biosensor,
wherein said acquisition unit interfaces with said injection and extraction module and said positioning and tracking module,
wherein said device for the insertion and extraction of a miniaturized biosensor is selected for one of manual and automated mode of operation.

2. The device of claim 1, wherein the said plunger is outfitted with a specialized tip.

3. The device of claim 2, wherein the specialized tip comprises at least one of a magnet and an electromagnet.

4. The device of claim 1, wherein the said array of imaging devices comprises from an array of magnetic-field detecting sensors.

5. The device of claim 1, wherein the said array of imaging devices comprises from an array of photodetectors.

6. The device of claim 5, wherein the said array of photodetectors is co-localized with an array of LEDs

7. The device of claim 6, where all said LEDs are activated to power the said miniaturized biosensor.

8. The device of claim 1, wherein the said catheter is outfitted with two magnets to facilitate capturing the implanted miniaturized biosensor.

9. The device of claim 1, wherein the said spring-actuated flap has a sharp edge capable to excise tissue.

10. A method for the implantation of a miniaturized biosensor underneath the skin comprising a microprocessor controlled:
injection module comprised of a catheter and a plunger that are interfaced with linear translation and rotary movement,
positioning and tracking module comprised of a moveable shaft, a multi-axes motorized stage, an array of imaging devices, and a temporary adhesive layer,
wherein said injection module houses said miniaturized biosensor within the catheter,
wherein said positioning and tracking module utilizes said temporary adhesive layer to adhere to the skin and said movable shaft to lift up the skin in a “π” shape form,
wherein said injection module utilizes the insertion catheter to pierce the skin,
wherein said positioning and tracking module tracks the said miniaturized biosensor within the catheter using the said array of imaging devices and guides the linearly translating catheter using the said multi-axes motorized stage at the desired depth and orientation with respect to the skin surface,
wherein said injection module retracts its said catheter, while holding fixed the plunger to position the said miniaturized biosensor at the proper depth and orientation,
wherein said injection module retracts the said plunger to release the said miniaturized biosensor at the proper depth and orientation underneath the skin.

11. The method of claim 10, wherein the said plunger is outfitted with a specialized tip to ensure the miniaturized biosensor is inserted with the proper orientation.

12. The method of claim 10, wherein the said array of imaging devices comprises from an array of magnetic-field detecting sensors.

13. The method of claim 12, wherein the said magnetic-field detecting sensors comprises from Hall effect sensors.

14. The method of claim 12, wherein the said magnetic-field detecting sensors comprises from giant magneto resistor sensors.

15. The method of claim 10, wherein the said array of imaging devices comprises from an array of photodetectors.

16. The method of claim 10, wherein the said array of photodetectors is co-localized with an array of LEDs

17. The method of claim 10 wherein the said plunger retraction and miniaturized biosensor release is facilitated by reversing the current polarity of an electromagnet.

18. The method of claim 10 wherein the said plunger retraction and miniaturized biosensor release is facilitated by a spring-actuated flap.

19. The method of claim 18, wherein the said spring-actuated flap is actuated by a flap-releasing rod that resides within the said catheter.

20. A method for the explantation of a miniaturized biosensor from underneath the skin comprising a microprocessor controlled:
extraction module comprised of a catheter, a plunger and a boring catheter that are interfaced with linear translation and rotary movement,
positioning and tracking module comprised of a moveable shaft, a multi-axes motorized stage, an array of imaging devices, and a temporary adhesive layer,
wherein said positioning and tracking module utilizes the said array of imaging devices to identify the position of the implant and align the extraction module in the proper position,
wherein said positioning and tracking module utilizes said movable shaft to lift up the skin in a “π” shape form,
wherein said injection module utilizes the insertion catheter to pierce the skin,
wherein said positioning and tracking module using the said array of imaging devices tracks the location of both catheter and implanted miniaturized biosensor and uses the said multi-axes motorized stage to line up the miniaturized implant with the linearly translated extraction catheter,
wherein said injection module utilizes the said boring catheter to excise the tissue around the said implanted miniaturizes biosensor and facilitate with its extraction,
wherein said injection module utilizes the said extraction catheter and plunger to capture and extract the said implanted miniaturizes biosensor.
